(19) United States
(12) Patent Application Publication
(10) Pub. No.: US 2025/0114190 A1
(43) Pub. Date: Apr. 10, 2025

(54) HEART VALVE PROSTHESIS

(71) Applicant: MITRASTREAM HEART VALVE TECHNOLOGY GMBH, Graben-Neudorf (DE)

(72) Inventor: Johannes JUNG, Sinsheim (DE)

(21) Appl. No.: 18/684,781

(22) PCT Filed: Aug. 19, 2022
(86) PCT No.: PCT/EP2022/073232

§ 371 Date: Jul. 22, 2024

(30) Foreign Application Priority Data
Aug. 19, 2021 (DE) 10 2021 121 556.0

(51) Int. Cl. A61F 2/24

(52) U.S. Cl. CPC A61F 2/2418; A61F 2/2427; A61F 2210/0014; A61F 2220/0008; A61F 2230/0004; A61F 2230/0034

(57) ABSTRACT

A heart valve prosthesis for minimally invasive intravenous implantation, has at least one movable valve leaflet made of a flexible, biocompatible material for opening and closing a through-opening between the atrium and ventricle of the heart, said movable valve leaflet secured to a holding element consisting of a stent-like cylindrical lattice structure made of a radially expandable material. The cross-section of the stent-like lattice structure has the shape of a kidney, a bean, or a banana. The heart valve prosthesis also alleviates ventricular dilation caused by elongation of the chordae.

### FIELD OF THE INVENTION

The invention relates to a heart valve prosthesis, in particular a mitral valve prosthesis, that is attached to a holding scaffold and that can be implanted via the transvenous route into the through-opening between the atrium and ventricle via a catheter in a minimally invasive procedure.

### BACKGROUND OF THE INVENTION

The incidence of acquired heart valve disease increases with age. Today, aortic stenosis is primarily treated via transcatheter aortic valve replacement (TAVR). The calcium deposits usually present at the native aortic valve create a high probability of anchoring of the TAVR valve.

Regurgitation (insufficiency) is the primary consideration in acquired mitral valve disease. However, the calcification of the mitral valve is generally insufficient for anchoring of an artificial heart valve. Furthermore, the mitral valve, which controls the flow of blood from the left atrium to the left ventricle, is difficult to access via minimally invasive procedures due to its position, as sharp angles/curves must be navigated, making catheter-based implantation very difficult. Furthermore, the function of the mitral valve is significantly more complex than that of the aortic valve.

Clinically significant regurgitation of the mitral valve (mitral valve insufficiency) affects approximately 8% of the population over age 75 in Western industrialized nations and, like aortic stenosis, is associated with significantly increased mortality (Nkomo V T, Gardin J M, Skelton T N, Gottdiener J S, Scott C G, Enriquez-Sarano M. Burden of valvular heart diseases: a population-based study. Lancet 2006; 368:1005-11). However, only 2% of patients undergo surgical treatment (Head S J, van Leeuwen W J, Van Mieghem N M, Kappetein A P. Surgical or transcatheter mitral valve intervention: complex disease requires complex decisions. EuroIntervention 2014; 9:1133-5). The high-risk nature of the surgical options and the lack of a simple and safe catheter-based valve replacement procedure are likely reasons for the insufficient treatment rate.

Most procedures used today are reparative procedures such as the MitraClip. A steep learning curve and low effectiveness are among the main limitations. Mitral valves designed for catheter implantation have been in development since 2012. Most systems attempt to transfer the technology successfully used for the aortic valve (TAVR) to the mitral valve, but the two valves have completely different structures and functions. Other disadvantages of these systems include requiring the transapical route (open surgery), requiring the initial insertion of an anchoring structure near the mitral annulus, causing left ventricular outflow tract obstruction, having leaflets that are too thick (optimized for the aortic valve), or altering the natural anatomy due to non-physiological designs. These factors all lead to increased complications during implantation and, even with technical success, to an increased risk of thrombotic events (Head S J, van Leeuwen W J, Van Mieghem N M, Kappetein A P. Surgical or transcatheter mitral valve intervention: complex disease requires complex decisions. EuroIntervention 2014, 9:1133-5.). The ideal system is implantable simply via a transvenous/transseptal approach, has a slim design that integrates with the natural anatomical features of the mitral annulus and holding apparatus, and has a slim valve, preferably bicuspid, with a flow profile similar to that of the natural mitral valve. Left ventricular outflow tract obstruction should be precluded if possible, which is a disadvantage of most of the concepts currently proposed.

Round, oval, or D-shaped cross-sections are described in the literature. None of these cross-sections enables the stent to precisely adapt to the natural shape of the mitral valve through-opening. For example, WO 2013/075215 describes a mitral valve prosthesis that can be implanted via catheter and consists of a D-shaped stent-like carrier ring with attached leaflets. The carrier ring has a D-shaped cross-section. This changes the natural valve apparatus from the physiological form.

US 2016/0331527 A1 discloses a heart valve prosthesis in which the heart valves are attached to a tube formed from wire with an inner and outer wall. The cross-section of the tube formed from wire can be a circle, kidney, or large D.

EP 3 456 293 A1 describes an implantation system for a heart valve prosthesis. In said system, the heart valves are attached to an expandable lattice anchor system that has a collar-shaped expanded radius on the atrial side.

### SUMMARY OF THE INVENTION

The invention therefore has the objective of providing a heart valve, in particular a mitral valve, that is simple and unproblematic to deploy and implant via the transvenous route. The invention also has the objective of simple alleviation of ventricular dilatation caused by elongation of the chordae.

Furthermore, there should preferably also be a very slim and flexible implantation device for implanting the heart valve prosthesis, wherein the device enables the prosthesis to easily be positioned and released, has the prosthesis already positioned in the correct anatomical orientation upon release from the catheter, and enables the prosthesis to orient itself. The heart valve prosthesis should preferably imitate the shape and function of the natural mitral valve and, in particular, should modify the natural chordae tendineae such that left ventricular reverse remodeling occurs. The heart valve prosthesis should preferably also not cause left ventricular outflow tract (LVOT) obstruction and should have with the two leaflets a flow profile corresponding to the natural flow profile to prevent thrombotic complications.

These objectives are achieved through the characteristics defined in the claims.

It is proposed according to the invention to attach a heart valve leaflet or a heart valve system to a stent-like holder, hereinafter also called a stent, with a shape optimally adapted to the anatomy of the heart, which can be introduced to the site of the heart valve and deposited there via implantation catheters optimized for this purpose.

According to the invention, it has proven advantageous for the cylindrical stent not to be perfectly circular, but rather to have a cross-section deviating from this in the direction of flow, the cross-section having the shape of a kidney, bean, or banana. The cross-section has a maximum length (a), a maximum width (b) and a minimum width (c), wherein (a) is greater than (b), and (b) is greater than (c). The length of (a) corresponds to the diameter of a circle circumscribing the heart valve.

The kidney shape, in particular the outer curvature, can best be described by a nearly circular circumference around the end point of the small width (c). In the simplest case, the curvature forms a circle with a center point inside the kidney shape at the end of the central small width (c), that is, in the recess at the deepest part of the kidney shape. This center point can deviate by +/−20% of the width (a), in particular +/−10%, preferably +/−5%. This may yield an oval or elliptical cross-section. If this is the case, the length (a) is divided unevenly, and the two parts are not the same length.

The preferred cross-section of a kidney shape according to the invention has a maximum length (a) that is typically 20-50 mm, in particular 25-40 mm, and preferably 28-38 mm and a large width (b) that is typically 10-30 mm and preferably 13-28 mm or 15-25 mm. The central small width (c) is typically 5-25 mm and preferably 7-23 mm or 9-20 mm. (a) is greater than (b), and (b) is always greater than (c).

Particularly preferably, the cross-section is the shape of two abutting D's, wherein one D is horizontally inverted, and wherein the straight vertical lines of the two D's abut and the edge of the cross-section is formed by different curvatures, or arcs, of the D's. In this shape, the length (c) defined in the aforementioned cross-sectional shapes is omitted and is replaced by the sum (d) of the respective maximum distances (maximum curvatures) of the two abutting D's, which is the sum of the large distance (maximum curvature) d₁ and the small distance (maximum curvature) d₂, each measured from the imaginary straight vertical line of the respective D. (a) is greater than (d). The distance d₁ (large curvature, or large arc) then corresponds to the length (b) of the aforementioned cross-sectional shapes. The curves merge without a recess where the curved lines of the two D's meet. In a special embodiment, this area can even be straight for a short length.

The aforementioned dimensions apply to the cross-sectional lengths (a) and d₁. For the smaller curvature of the smaller D, the maximum distance d₂ from the imaginary vertical stroke/straight line of the D is at least 0.5 mm, preferably at least 0.75 mm, wherein at least 1 mm, in particular at least 2.5 mm, or 3.0 mm, is particularly preferred. In an appropriate embodiment, the curvature is at least 7.5 mm. The ratio of the two lengths of the imaginary straight line (d) between the large d₁ (smaller radius) and the small d₂ (larger radius), that is, d₁ to d₂, is at most 20:1, in particular at least 2:1, wherein ratios of at most 5:1, or at most 10:1, and in particular at most 15:1, are preferred. The height of the line (a) forming the respective imaginary back of the D is 15 mm to 50 mm, in particular 20 mm to 45 mm, based on the desired size of the heart valve.

In a further preferred embodiment, the stent has a smaller web width on the side of the smaller curvature with the distance d₂ (large radius) than on the side of the larger curvature (with the distance d₁). The web side on the larger curvature is typically 0.3 mm to 0.6 mm, but preferably at least 0.35 mm and at most 0.4 mm or 0.55 mm. This web side is thinner on the side of the flatter curvature (large radius) and is at most 55% of the web width on the side with the larger curvature. In a preferred embodiment, the web width is at least 10%, in particular 20% or 25%, of the web width on the more sharply curved side (on the right in FIGS. 8a and 8b).

With the prosthesis according to the invention, due in particular to the different thicknesses of the stent webs, it is possible for the heart valve prosthesis inserted to adapt to the beating heart, in particular near the stent. It has specifically been shown that the embodiment with the thinner/narrower web width enables the moving heart muscle and the stent prosthesis to move together during contraction and does not remain particularly rigid from the strong resistance of the stent. This enables the stent to adapt to the natural movement of the heart muscle and to securely seal the through-opening even in its movable form.

Preferably, the cross-section of the stent-like lattice structure first decreases in the direction of flow from the atrium and increases again after the through-opening to the ventricle, creating a somewhat waist-shaped constriction surrounded by the heart muscle. This causes the stent-like lattice structure or scaffold of the prosthesis to sit securely in the through-opening of the heart.

The cross-sections shown in FIGS. 6a, 6b and 6c are the same object shown from different sides and form different perspectives including different features. The cross-sections are preferably of a cylinder constricted on the atrium side, represented by two separate truncated cones. The height of the upper truncated cone is 51-80% of the total length of the stent, and that of the lower truncated cone is 49-20% of the total length of the stent.

The shape of the stent-like valve leaflet holder according to the invention adapts to the natural shape of the mitral valve, deviating significantly from a circle and forming the shape of a bean, kidney, or banana in cross-section, which can be symmetrical or asymmetrical in all cross-sectional planes (relative to the direction of flow).

In a preferred embodiment, the stent has a dimension, or area, near the through-opening between the atrium and ventricle that is larger than the natural environment, whereby any dilated chordae are re-tensioned.

In a further preferred embodiment, the stent-like lattice structure or the holding device has small outwardly directed holding elements or barbs on the side facing the atrium and/or on the side facing the ventricle. This enables secure anchoring of the stent-like holding element to the valve opening.

FIG. 1 shows a stent with circumferential atraumatic holding elements. The number of holding elements is between 2 and 15, typically at least 4, preferably at least 8.

The valve leaflets are attached to the holding element, or the stent-like holding device and preferably are made of a biocompatible material, such as a biological tissue from collagen, an autograft, a human graft or a zenograft.

Near the natural posterior leaflet, the stent, or the heart valve holder, or heart valve scaffold has a curve with different curve radii (PML; P1 segment to the anterior commissure, P2 middle segment of the PML, and P3 segment to the posterior commissure) (FIG. 7), wherein the curve radii can differ from each other. In particular, the P1 and P3 segments do not exhibit complete mirror symmetry. The curvature near the P2 segment has the largest radius and P1 and P3 have a smaller radius, wherein, according to the invention, the radius of P1 can be less than, greater than, or equal to P3.

In a further preferred embodiment, the shape of the heart valve holder near the anterior mitral leaflet (AML) is adapted to the natural anatomy of the mitral valve.

Relative to the imaginary curve arcing toward the native PML, which spans between the commissures of the natural mitral valve, the shape of the stent, or the heart valve holder, or heart valve scaffold, at the segments A1 and A3 near the commissures curves outward toward the LVOT, which then crosses the imaginary curve between the commissures inward toward the native PML in the middle section of the A1 and A3 segments or in the respective transition to the A2 segment and continues this in an arc. The respective curve radii are typically not symmetrical between the A1 and A3 segments, and the curve shape within the A2 segment also does not have mirror symmetry. The respective radii can be larger, smaller, or equal.

The physiological function of the mitral valve also includes movement in the third dimension based on the cardiac cycle. As such, the mitral valve presented here enables deformation during the cardiac cycle at the level of the mitral annulus and in the direction of the left atrium and left ventricle. Adaptation to the natural anatomy of the native mitral valve also enables better fixation and better retention of the prosthesis. Furthermore, this shape has been shown to be self-centering.

With the prosthesis according to the invention, the natural holding apparatus of the valve remains intact, as the prosthesis is secured in the natural valve opening, and without significantly altering other anatomical characteristics. In addition, it has proven particularly useful to select a stent to which the actual valve leaflets are attached that is larger than the previous diameter of the through-opening. This enables re-tensioning of dilated or overstretched chordae that hold the actual valves.

This also results in increased pretension of the natural chordae tendineae and their associated papillary muscles so that in the best case, left ventricular reverse remodeling occurs. This is facilitated by the shape and function of the valve prosthesis according to the invention, which imitates the natural mitral valve and thus yields uniformly better pretension of the natural chordae tendineae in all planes.

Left ventricular outflow tract obstruction is always precluded, even if the anatomy is unfavorable for this, because the position is adapted to the natural anatomy.

The valve leaflets are typically bicuspid, but valve prostheses with 3 or even 4 individual leaflets could also be used.

Holding chordae integrated into and attached to the valve scaffold imitate the natural chordae tendineae and prevent the leaflets from penetrating the atrium when the ventricle contracts. The number of holding chordae per leaflet is between 1 and 15, typically at least 3, preferably at least 4. The maximum number of holding chordae is 15, preferably a maximum of 9 or 7.

The valve leaflets form an adaptation line in their contact surface, along which the valve leaflets abut each other and seal off/prevent regurgitation. The leaflets' adaptation line typically corresponds to the shape of the stent, but it can also deviate from this. Typically, the adaptation line extends from the natural A1/P1 commissure to the natural A3/P3 commissure, but it can also be shifted anteriorly or posteriorly by up to 5 mm, in particular 4 mm or 3 mm. The curvature of the adaptation line follows the adaptation line of the natural valve, but it can also be shifted anteriorly or posteriorly by up to 5 mm, in particular 4 mm or 3 mm. In a preferred embodiment, the leaflets continue on the atrial side of the inside of the stent toward the ventricle for lateral sealing, as shown in FIG. 6b, for example.

To insert such a heart valve system, it must first be loaded into a delivery system/catheter system, which is introduced via the transvenous route, either via an anatomically relatively caudal vein (typically, but not necessarily, percutaneously via the femoral vein or via veins located in the pelvis accessed surgically), or a relatively cranial vein (typically, but not necessarily, percutaneously or surgically via a jugular vein or the subclavian or axillary vein). The delivery system has the necessary dimensions and especially the flexibility to follow the path to the mitral valve by transseptal access from the right into the left atrium and onward, toward the left ventricle through the natural mitral valve via a previously placed guidewire, even if the distance between the transseptal puncture level and the mitral valve level is typically not more than 4 cm, typically not more than 3 cm, and preferably not more than 2 cm.

Controllable catheters are well suited to position the prosthesis according to the invention. The distal end of such catheters can be bent into an arc. This arc then forms a plane with the vein through which the system is fed, which is also bent. From the perspective of the cross-section of the vein, the outside of the arc, that is, the side facing the other ventricle (right ventricle), is at the 12:00 position, and the point on the inside of the arc is at the 6:00 position.

The delivery system is slim and flexible, and the bend of the distal section can be controlled. The size of the catheter shaft is a maximum of 14 French and is typically between 8 and 12 French. The distal part of the delivery system carries the stent with the valve secured inside it, the valve having a circumference only a few millimeters larger than the catheter shaft, typically 2 mm, also between 0.5 mm and 4 mm. The arc of the catheter shaft, or the distal part of the delivery system, can be controlled, typically between −20 to about 210 degrees. The delivery system enables the valve to be loaded in a previously defined orientation, brought to its destination, and released in a defined anatomical position.

In a preferred embodiment, the stent of the prosthesis according to the invention has a positioning aid such that the application can only be loaded for delivery at a location provided for this purpose. The positioning aid only fits into the system at this location, like a key in a keyhole. It has proven beneficial to orient the positioning aid from 6:00-8:00, preferably from 6:30-7:30, based on the aforementioned position. In addition, it has also proven beneficial to place radiopaque markings and/or markings detectable via ultrasound on the stent, which enable the operating physician to check the position of the stent and thus the valve leaflets.

### BRIEF DESCRIPTION OF THE DRAWINGS

The invention is explained by way of example in the following figures.

The following are shown:

FIG. 1 shows top and bottom views of a stent or stent-like holder, having a lattice or web structure, comprising securing elements (1.1) and circumferential atraumatic holding elements (1.3);

FIG. 2 shows a top view, or cross-section, of the stent-like holding lattice of FIG. 1 showing a centering (2.1) eyelet at the 7:00 position and positioning (2.2) elements;

FIG. 3 shows a catheter system for implanting or delivering the heart valve, showing a loaded stent implant (3.3) held within the catheter on a stent holder (3.2);

FIG. 4 shows a partially released stent/implant;

FIG. 5a shows top views of a stent-like holding element in the direction of blood flow;

FIG. 5b shows a side view of the stent-like holding element having a web or lattice holding structure;

FIG. 6a shows a section of the mitral valve prosthesis showing the stent holder (1.0) and valve leaflets (6.1);

FIG. 6b shows the valve leaflets continuing on the atrial side of the inside of the stent toward the ventricle for lateral sealing;

FIG. 6c shows the adaptation line between A1/P1 to the location of the natural commissure between A3/P3;

FIG. 7 shows the segmental anatomy of a natural mitral valve with the position of the anterior A1-A3 commissure and the posterior P1-P3 commissure; and

FIG. 8a shows a particularly preferred configuration of an embodiment of the web of the heart valve prosthesis; and

FIG. 8b shows the preferred shape of the stent cross-section of the heart valve prosthesis.

### DETAILED DESCRIPTION OF THE INVENTION

The invention is direct to a heart valve prosthesis, comprising a valve prosthesis and a holding element for the prosthesis. The valve is typically a bicuspid (two leaflets) valve, but valve prosthesis having three or even four leaflets are within the scope of the invention.

The invention is best described in detail by reference to the figures and the explanations thereof contained herein.

With respect to the invention, terms such as “valve”, “valve prosthesis”, “heart valve”, “heart valve leaflet”, “heart valve system”, or “valve leaflets” are synonymous and refer to a heart valve that is supported or held within a holding structure for implantation into the heart.

The terms “stent”, “stent holding element”, “stent-like”, “stent scaffold”, “stent-like lattice”, “lattice”, “web”, “valve leaflet holder”, “heart valve holder or holding element”, “heart valve scaffold” are synonymous and refer to the support or holding structure within which the valve prosthesis is placed for implantation into the heart.

“Catheter” or “implantation device” is the device in which the heart valve prosthesis is loaded and then guided and placed into the heart, followed by release.

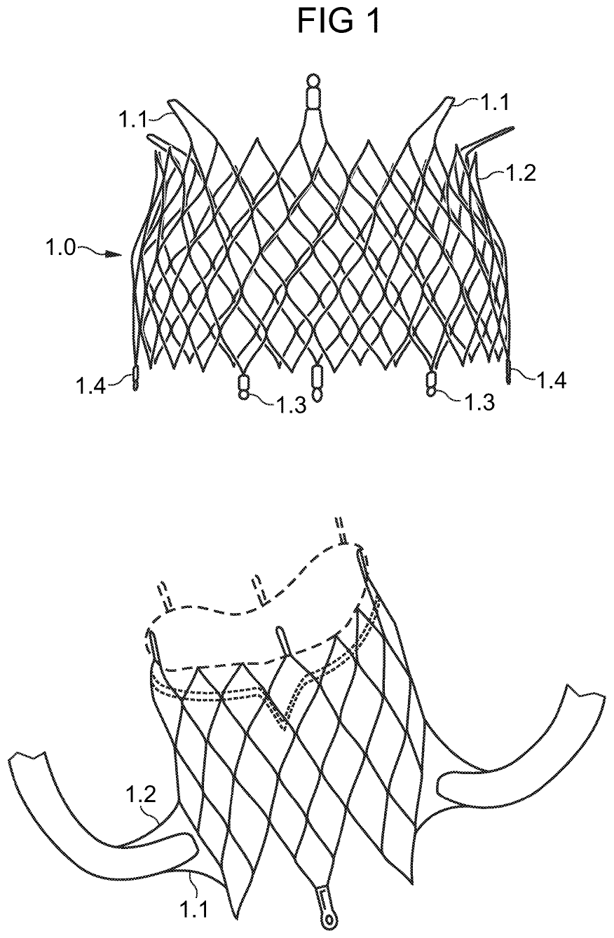

FIG. 1 shows a stent-like heart valve holder, or heart valve holding element, 1.0. The stent-like holder, or lattice structure, has a securing element 1.1 for securing the stent-like element in the heart. Such a securing element 1.1 is preferably in the form of an eyelet. The eyelet itself curves outward from the center axis (direction of blood flow) of the stent-like lattice element so that it anchors itself in the heart muscle. It also has a constriction 1.2 around its cylindrical circumferential surface. Along this constriction 1.2, the stent, or the stent-like lattice structure, has a smaller circumference than at its two distal ends. At the distal end opposite the securing elements, the lattice structure of the holder has holding eyelets 1.3 for securing the holder at a location provided for this purpose in the delivery system, or catheter, wherein at least one holding or retaining element has a marker 1.4 to enable checking of the location of a valve holder, or stent, opened in the heart. Such markers are preferably radiopaque. It is also theoretically possible for such markers to be made of materials that are particularly easy to see via ultrasound.

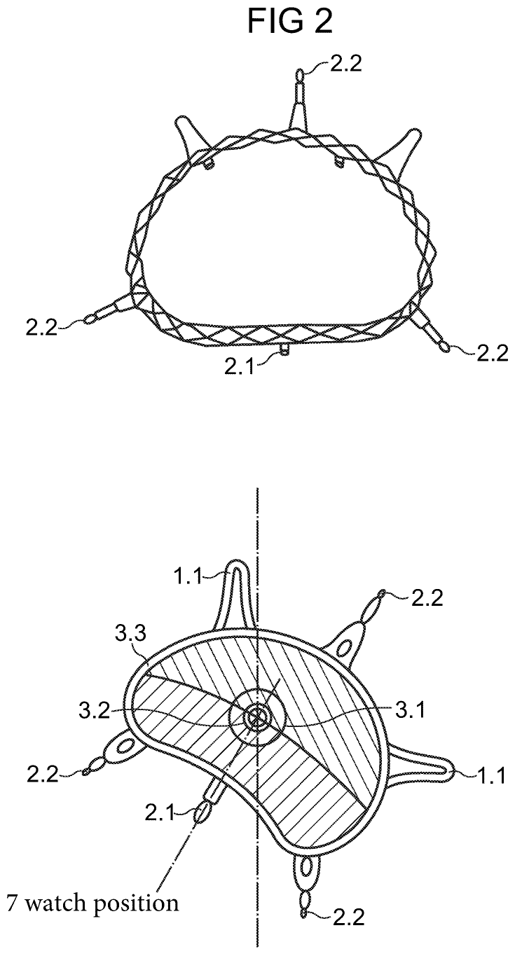

FIG. 2 shows a front view, or cross-section, of the stent-like holding lattice, or element, of FIG. 1. As can be seen from this, such a stent is preferably not perfectly round, but rather is flat on at least one side. This enables it to adapt to the natural shape at the through-opening in the heart between the atrium and the ventricle.

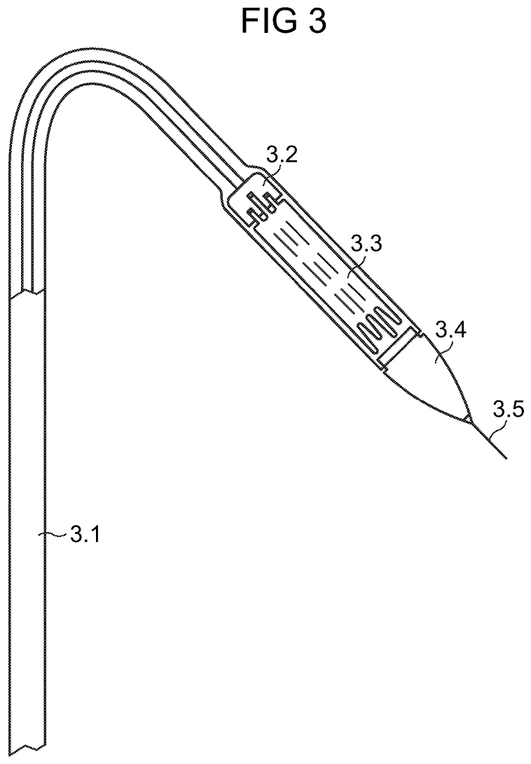

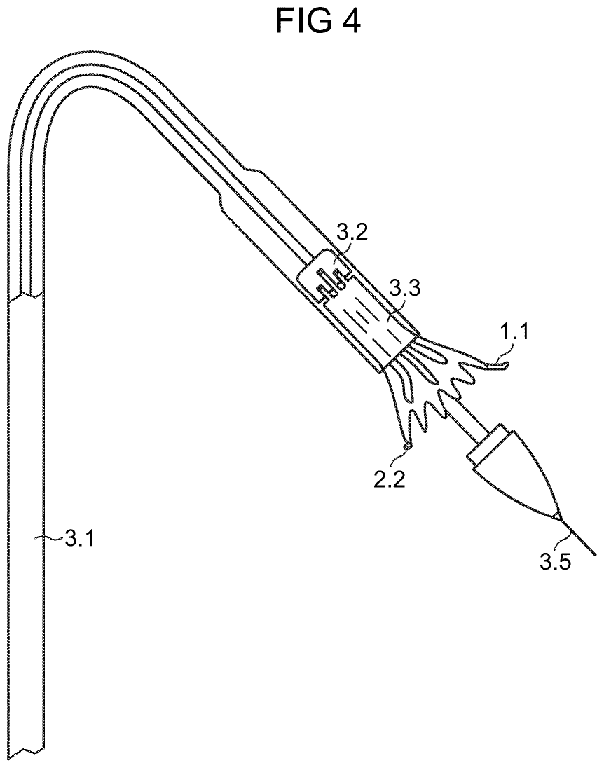

FIGS. 1 and 2 show the essential structure/shape of the stent scaffold of the valve. The shape is adapted to the natural shape of the natural mitral valve. There are preferably one or more retaining eyelets 1.4 on the atrial side for loading the prosthesis, which enable only a predefined position in the holding system of the delivery system/catheter system (FIGS. 3, 4) and enable retraction of the heart valve back into the delivery system until shortly before release if necessary for retrieval or repositioning or if final release is not yet desired. The retaining eyelet(s) is/are held in place on the catheter via positive mechanical engagement. There is also a constriction 1.2 of the stent-like heart valve holder at the level of the native mitral annulus, which enables a defined height and provides stability in the anatomically correct position. The ventricle side has round formations, or securing elements 1.1, for the stent, which also ensure the correct anatomical height and stability of the system.

FIG. 3 shows a catheter system for placing the heart valve according to the invention with a loaded stent/implant 3.3 held in the catheter system 3.1 by the stent holder 3.2. The catheter system is closed off by a flexible tip 3.4 at the front and is guided by a guidewire 3.5.

The catheter system 3.1 and the holder for the stent-like holding scaffold 3.2 are therefore preferably designed such that the 7:00 position in the top view of the catheter tip has a recess to accommodate the centering element, or the centering eyelet, 2.1, so that the stent-like holding element can only be loaded in the catheter in this orientation. The centering element (eyelet) 2.1 is on the stent/implant 3.3. The centering element has two functions in particular, namely first to load the stent only in the correct position, and second to secure the stent-like holder, or the entire implant. The holding eyelets 1.3 and 1.4 on the stent-like holding scaffold 1.0 also secure the prosthesis to the holder 3.2 so the stent can be released, or positioned, evenly and without “jumping.” The holding eyelets (1.3 or 1.4) may also have markers, which show the position and orientation of the stent after positioning and enable checking of the opened stent/implant.

The defined anatomical position largely corresponds to the natural shape of the mitral valve. When the valve is released, it reorients by −30 to +30 degrees, typically between 15 and 25 degrees, and in particular up to 20+/−3 degrees, to the natural anatomy.

The orientation of the catheter system relative to the native mitral valve is defined by the plane that is defined by the curved catheter system perpendicular to the atrial septum and that also intersects the valve plane perpendicularly. This virtual plane intersects an imaginary clock on the mitral valve at 12:00 and 6:00. The position of the stent with the valve is rotated 30 degrees to the right in the catheter so that the position marker points to the 7:00 position (FIG. 2).

FIG. 4 shows a partially released stent/implant 3.3 in which the positioning eyelets 2.2 show the position, or orientation, of the stent/implant via fluoroscopy. The positioning eyelets indicate the correct position of the stent/implant. The positioning eyelets also function as holding eyelets; that is, the holding eyelet acts as an end stop at the old valve. The securing eyelets 1.1 also secure the stent/implant where it was deposited.

The horizontal cross-section of the valve holder 1.0 according to the invention preferably has the shape of a bean or kidney. This shape and loading in the catheter system 3.1 in the 7:00 position cause the valve holder (stent/implant) 1.0 to already have the predetermined optimal orientation in terms of position and location in the heart when it is released from the catheter system 3.1.

FIG. 2 shows the top view of the stent/implant 3.3; here, the centering eyelet 2.1 is clearly visible in the 7:00 position.

The provisions according to the invention of the predefined loading in the delivery system, the controllable and defined bending behavior of the delivery system, the orientation of the valve plane to the delivery system, and the self-aligning properties of the valve (rotation and height), enable implantation with conventional imaging procedures and the generally expected expertise of a radiographer. Conventional X-ray fluoroscopy with a coronary angiogram or right heart ventricular angiography or levocardiography and transthoracic as well as transesophageal echocardiography in two-dimensional or multidimensional resolution are sufficient for imaging. Intracardiac echocardiography (ICE) can also be used.

It has also proven beneficial to apply various additional positioning aids. It has also proven advantageous to design these positioning aids differently so that during implantation, the operating physician can determine at any time how to safely and easily rotate the stent-like holder into the correct orientation for implantation.

Such positioning aids are, for example, radiopaque. It is also theoretically possible to deploy them with other media that are clearly visible on an ultrasound, for example.

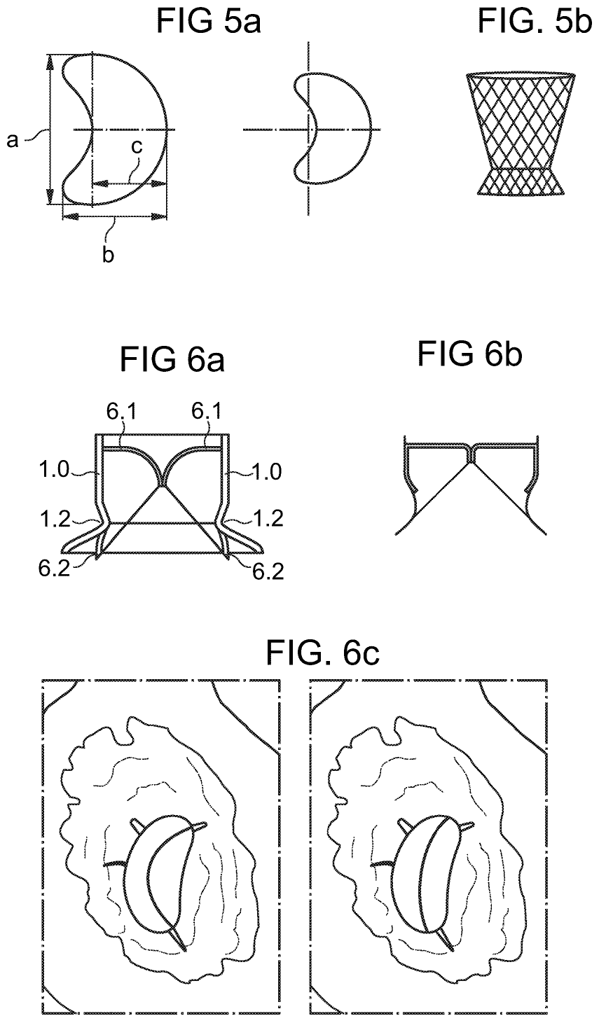

FIG. 5a shows a stent-like holding element in the shape of a bean, kidney, or banana in a top view in the direction of blood flow and in a side view (FIG. 5b) showing the web or lattice structure of the stent-like holding element.

This side view shows a stent-like holding element with two opposing funnels, which has a constriction 1.2 at the narrowest point. (FIG. 1) This constriction centers and positions the stent in the plane of the valve. The constriction also locks down the stent/implant 3.3, thereby securing it upward and downward.

FIG. 6a shows a section of the mitral valve prosthesis (1.0) according to the invention with valve leaflets 6.1, the chordae or chordae tendineae 6.2 holding the leaflets, and the constriction 1.2.

FIG. 6b shows the valve leaflets continuing on the atrial side of the inside of the stent facing toward the ventricle for lateral sealing.

FIG. 6c shows the adaptation line between A1/P1 to the location of the natural commissure between A3/P3.

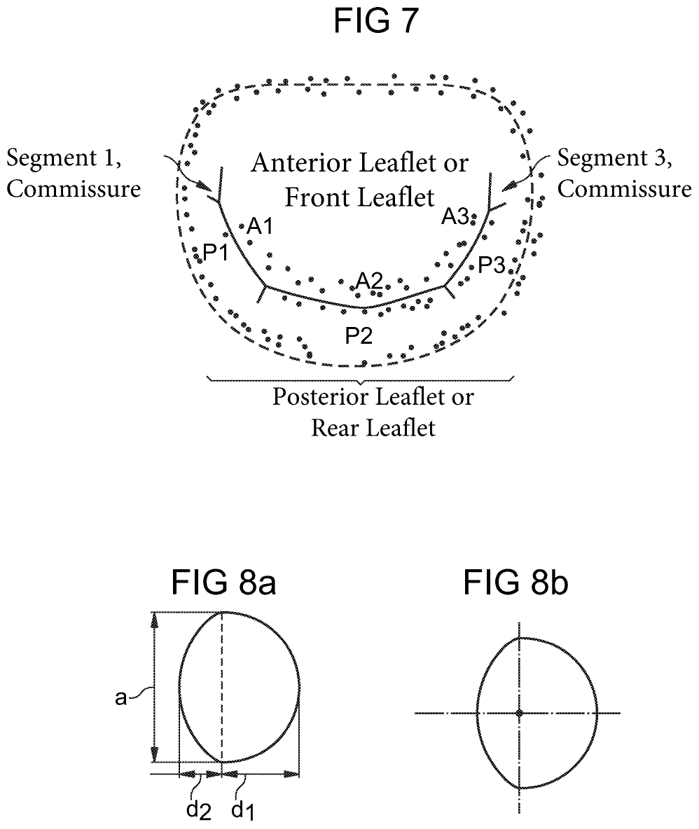

FIG. 7 shows the segmental anatomy of a natural mitral valve with the position of the anterior A1-A3 commissure and the posterior P1-P3 commissure.

FIG. 8a shows a particularly preferred embodiment of the web or lattice of the heart valve prosthesis according to the invention. As can be seen from this, the cross-section is formed by two shapes lying opposite each other on their vertical sides, each forming a large D. The larger part of the cross-section with the large D is a maximum distance d₁ from the imaginary straight line of the D. The arc of the smaller D on the left is a maximum distance d₂ from the imaginary straight line. The total width of the stent (from the maximum D curvature on the left side to the maximum D curvature on the right side) is therefore d₁+d₂. The maximum length of the D (corresponding to the imaginary vertical line of the letter D) is represented by the length (a). FIG. 8b shows the preferred shape of the stent cross-section of the heart valve prosthesis according to the invention. The shapes shown in FIGS. 8a and 8b have a smaller web width, particularly on the left side.

### REFERENCE DESIGNATION LIST

1.0 Heart valve holder
1.1 Securing elements
1.2 Constriction
1.3 Holding or retaining eyelets
1.4 Holding or retaining eyelets
2.1 Centering eyelet
2.2 Positioning eyelets
3.1 Catheter system
3.2 Holding frame
3.3 Implant (stent lattice and leaflets)
3.4 Catheter tip
3.5 Guidewire
6.1 Mitral valve leaflets
6.2 Mitral valve chordae

What is claimed is:

1. A heart valve prosthesis for minimally invasive intravenous implantation, comprising:
at least one movable valve leaflet comprised of a flexible biocompatible material for opening and closing a through-opening between the atrium and ventricle of the heart,
wherein the movable valve leaflet is secured to a holding element consisting of a stent-like lattice structure comprising a radially expandable material,
wherein the cross-section of the stent-like lattice structure has the shape of a double-D formed by two abutting D's, wherein one D is horizontally inverted and straight vertical lines of the two D's abut to form an imaginary straight line.

2. The heart valve prosthesis according to claim 1, wherein the edge of the cross-section formed by the double D shape is formed by the different curvature or arcs of the two D's and, wherein one D has a greater curvature and the other D has a smaller curavature.

3. The heart valve prosthesis according to claim 2, wherein a ratio of a maximum distance between the D having a greater curvature from the imaginary straight line and a corresponding maximum distance of the other D having a smaller curvature from the imaginary straight line is 20:1 mm to 2:1 mm.

4. The heart valve prosthesis according to claim 2, wherein the stent-like lattice structure on the side of the D with the smaller curvature has webs with a smaller web width than the webs on the stent side of the D with the greater curvature.

5. The heart valve prosthesis according to claim 1, wherein the stent-like lattice structure has circumferential atraumatic holding elements for securing the stent-like lattice structure to the heart.

6. The heart valve prosthesis according to claim 1, wherein the stent-like lattice structure has a positioning aid.

7. The heart valve prosthesis according to claim 1, wherein a cross-section of the stent-like lattice structure has a maximum length of 20-50 mm.

8. The heart valve prosthesis according to claim 1, wherein the biocompatible material is a biological tissue from collagen, an autograft, human graft, or xenograft.

9. The heart valve prosthesis according to claim 1, wherein the length of the lattice structure is selected such that it projects at least 2 mm into the atrium.

10. The heart valve prosthesis according to claim 1, wherein the stent-like lattice structure is made of nitinol.

11. The heart valve prosthesis according to claim 1, wherein the stent-like lattice structure has outwardly directed securing elements.

12. The heart valve prosthesis according to claim 1, wherein the cross-section of the stent-like lattice structure decreases from the atrium to the through-opening and increases again in the direction of flow after the through-opening.

13. The heart valve prosthesis according to claim 1, wherein the prosthesis comprises two movable valve leaflets that are miter-shaped bicuspid valve leaflets.

14. An implantation device for implanting a heart valve prosthesis according to claim 1, wherein the implantation device comprises a controllable catheter having an element exclusively for reception of a positioning aid on the stent-like lattice structure.

15. The implantation device according to claim 14, wherein the positioning aid on the stent-like lattice structure is at or near the 7:00 position.

16. A mitral valve prosthesis for minimally invasive intravenous implantation, comprising:
a. a holding element consisting of a stent-like structure comprising a radially expanding material; and
b. two movable, miter shaped bicuspid valve leaflets contained within the stent-like structure for use in opening and closing a through-opening between an atrium and ventricle of a human heart;
wherein the stent-like structure has a cross section that decreases from the atrium to the through-opening and increases again in the direction of flow after the through-opening,
wherein the stent-like structure has a shape of a double-D formed by two abutting D's, one D is horizontally inverted so that the straight vertical lines of the two D's abut to form an imaginary straight line,
wherein one 0 has a greater curvature than the other D having a smaller curvature, and
wherein a ratio of a maximum distance between the imaginary straight line to the greater curvature D and a corresponding maximum distance between the imaginary straight line and the smaller curvature D is 20:1 to 2:1 mm.

17. The mitral valve prosthesis according to claim 16,
wherein the stent-like holding structure is a lattice or web structure having outwardly directed securing elements, circumferential atraumatic holding elements for securing the prosthesis to the diseased mitral valve, and a positioning aid corresponding to a receiving portion of an implantation device, and
wherein the stent-like holding structure is made of nitinol.

18. The mitral valve prosthesis according to claim 16, wherein the two movable, miter-shaped bicuspid valve leaflets are made of a biocompatible material consisting of a biological tissue from collagen, an autograft, human graft or xenograft.

19. The mitral valve prosthesis according to claim 17, wherein the implantation device is a controllable catheter having a receiving portion for the positioning aid in the 7:00 position.
